(19) United States
(12) Patent Application Publication
(10) Pub. No.: US 2006/0110334 A1
(43) Pub. Date: May 25, 2006

(54) Two-compartment aerosol device comprising electrophilic monomers, and use thereof for the cosmetic treatment of the hair

(76) Inventor: Isabelle Rollat-Corvol, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/248,334

(22) Filed: Oct. 13, 2005

Related U.S. Application Data
(60) Provisional application No. 60/646,484, filed on Jan. 25, 2005.

(30) Foreign Application Priority Data
Oct. 13, 2004 (FR) 04 10809

(51) Int. Cl. A61K 8/81; A61K 9/00

(52) U.S. Cl. 424/47

(57) ABSTRACT

A two-compartment aerosol device comprising: (i) a first compartment comprising a cosmetic composition comprising, in at least one cosmetically acceptable medium, at least one electrophilic monomer, and (ii) a second compartment comprising at least one compressed gas, wherein the first and second compartments provide, during the use of the aerosol device, a permanent mechanical separation therein between the cosmetic composition and the at least one compressed gas and methods of using the two-compartment device.

This application claims benefit of U.S. Provisional Application No. 60/646,484 filed Jan. 25, 2005, the contents of which are incorporated herein by reference. This application also claims benefit of priority under 35 U.S.C. §119 to French Patent Application No. 04 10809 filed Oct. 13, 2004, the contents of which are also incorporated by reference.

Disclosed herein is a two-compartment aerosol device comprising at least one electrophilic monomer and at least one compressed gas, and the use of the cosmetic composition contained in the aerosol device for the cosmetic treatment of keratin materials, for example, hairstyling.

Hairstyling compositions, such as lacquers and sprays, packaged in aerosol spray form, may generally be composed of a liquid phase comprising, in an aqueous, alcoholic, or aqueous-alcoholic cosmetically acceptable medium, at least one fixing material and a propellant, which is a liquefied gas under reduced pressure or dissolved in the liquid phase.

Other aerosol devices comprising such a liquid phase may exist, but they comprise compressed gases to propel the liquid phase. However, these compressed-gas devices may have the drawback of losing gas pressure over time. The reason for this is that the compressed gases may leak over time or the devices may be used incorrectly, i.e. the devices may be turned upside down.

This drawback may result in increasingly poor propulsion of the liquid phase over time and, consequently, increasingly poor distribution of the liquid phase onto the hair.

Another factor that may lead to poor distribution of the liquid phase from these devices is the quality of the spray. For example, the spray may degrade when it is desired to obtain the best possible fixing, i.e., when it is sought to increase the amount of polymer that can be dissolved in the liquid phase.

In the field of hairstyling, the fixing materials are generally fixing polymers, i.e., film-forming polymers that are soluble or dispersible in water and in alcohol, such as vinyl acetate/crotonic acid copolymers, anionic or amphoteric acrylic resins, and polyurethanes. These polymers, when mixed with a suitable solvent, constitute the liquid phase in an aerosol container, which allows them to be sprayed onto the hair in the form of a cloud of dispersed droplets.

Once applied to the hair, the liquid phase dries, allowing for the formation of welds required for the fixing of the hairstyle by the fixing materials. The welds must be strong enough to ensure hold of the hairstyle.

However, the welds must also be fragile enough for the user to be able to destroy them by combing or brushing the hair, without damaging the scalp or the hair.

The present inventors have discovered, surprisingly, that the mechanical separation of the liquid phase from the compressed gas providing the propulsion, and the use of electrophilic monomers as described in Patent Application No. FR 2 840 208, may make it possible to solve at least one of the problems of leakage and of distribution of the liquid phase on keratin materials such as the hair and also may result in improved fixing and hold of the hairstyle, i.e., allowing the user to obtain welds of better quality than those obtained with a standard fixing material, while at the same time maintaining at least some good cosmetic properties such as softness and disentangling.

Disclosed herein is thus a two-compartment aerosol device comprising a cosmetic composition that comprises at least one electrophilic monomer as described below, in a first compartment, and at least one compressed gas in a second compartment, wherein the first and second compartments provide, during the use of the device, a permanent mechanical separation therein between the cosmetic composition and the at least one compressed gas.

Further disclosed herein is the use of the composition contained in the device for the cosmetic treatment of keratin materials, for example, for styling hair.

Even further disclosed herein is a hair treatment process comprising spraying the disclosed composition as defined herein onto the hair, in the presence of at least one nucleophilic agent as defined herein.

The spraying onto the hair of a cosmetic composition that comprises at least one electrophilic monomer, using such a device, may lead to a uniform distribution of the product on the hair and to at least some good cosmetic effects.

Other subjects, characteristics, aspects and advantages of the various embodiments disclosed herein will emerge even more clearly on reading the description and the examples that follow.

The two-compartment aerosol device disclosed herein comprises:

(i) a first compartment comprising a cosmetic composition comprising, in at least one cosmetically acceptable medium, at least one electrophilic monomer and

(ii) a second compartment, comprising at least one compressed gas, wherein the first and second compartments provide, during the use of the device, a permanent mechanical separation therein between the cosmetic composition and the at least one compressed gas.

The at least one compressed gas may be chosen, for example, from air, nitrogen, carbon dioxide, and mixtures thereof. In one embodiment, the at least one compressed gas is air.

The at least one compressed gas may, for example, be used at a pressure ranging from 0.1 to 1.2 MPa and, further, for example, from 0.9 to 1.1 MPa.

An electrophilic monomer is a monomer capable of undergoing polymerization by anionic polymerization in the presence of a nucleophile such as the hydroxyl (OH⁻) ions present in water.

“Anionic polymerization” as used herein refers to the mechanism defined in the work “Advanced Organic Chemistry”, Third Edition, Jerry March, pages 151 to 161.

The at least one electrophilic monomer present in the composition disclosed herein may be chosen from:

(i) benzylidenemalononitrile derivatives (A), 2-(4-chlorobenzylidene)malononitrile (A1), ethyl 2-cyano-3-phenylacrylate (B) and ethyl 2-cyano-3-(4-chlorophenyl) acrylate (B1), as described in Sayyah, J. Polymer Research, 2000, p. 97:

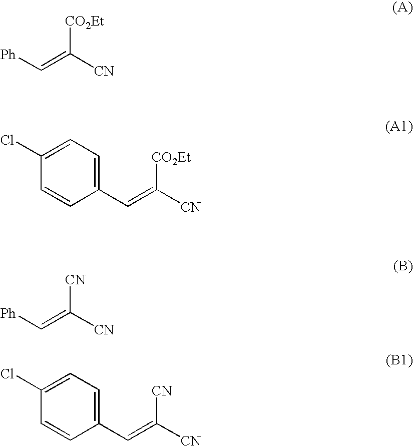

(ii) methylidenemalonate derivatives such as:

diethyl 2-methylenemalonate (C) as described by Hopff, Makromolekulare Chemie, 1961, p. 95, by De Keyser, J. Pharm. Sci, 1991, p. 67, and by Klemarczyk, Polymer, 1998, p. 173:

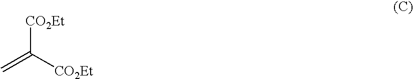

ethyl 2-ethoxycarbonylmethyleneoxycarbonylacrylate (D), as described by Breton, Biomaterials, 1998, p. 271 and Couvreur, Pharmaceutical Research, 1994, p.1270:

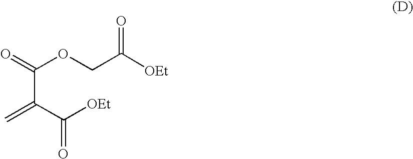

(iii) itaconate and itaconimide derivatives such as:

dimethyl itaconate (E), as described by Bachrach, European Polymer Journal, 1976, p.563:

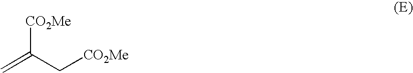

N-butylitaconimide (F), N-(4-tolyl)itaconimide (G), N-(2-ethylphenyl)itaconimide (H) and N-(2,6-diethylphenyl)itaconimide (I), as described by Wanatabe, J. Polymer Science: Part A: Polymer Chemistry, 1994, p. 2073:

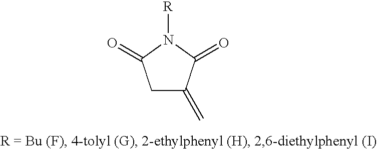

(iv) methyl α-(methylsulphonyl)acrylate derivatives (K), ethyl α-(methylsulphonyl) acrylate derivatives (L), methyl α-(tert-butylsulphonyl)acrylate derivatives (M), tert-butyl α-(methylsulphonyl)acrylate derivatives (N) and tert-butyl α-(tertbutylsulphonyl)acrylate derivatives (O), as described by Gipstein, J. Org. Chem, 1980, p. 1486, and 1,1-bis(methylsulphonyl)ethylene derivatives (P), 1-acetyl- 1-methylsulphonylethylene derivatives (Q), methyl α-(methylsulphonyl)vinylsulphonate derivatives (R) and α-methylsulphonylacrylonitrile derivatives (S), as described by Shearer, U.S. Pat. No. 2,748,050:

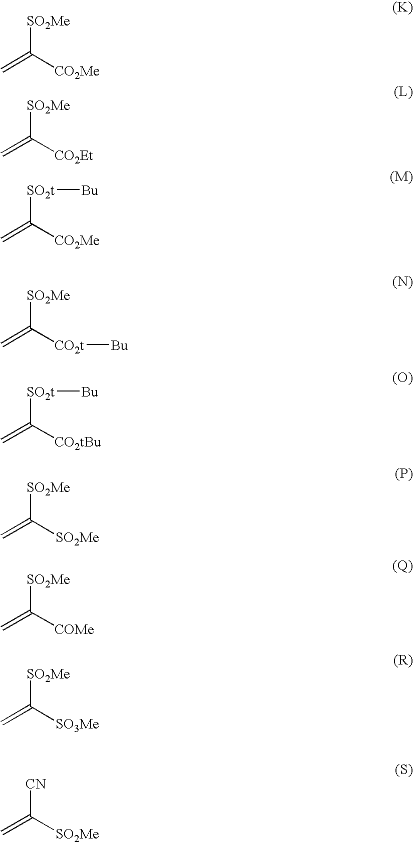

(v) methyl vinyl sulphone derivatives (T) and phenyl vinyl sulphone derivatives (U), as described by Boor, J. Polymer Science, 1971, p. 249:

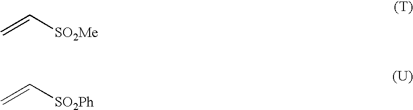

(vi) the phenyl vinyl sulphoxide derivative (V), as described by Kanga, Polymer Preprints (ACS, Division of Polymer Chemistry), 1987, p. 322:

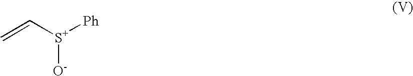

(vii) the 3-methyl-N-(phenylsulphonyl)-1-aza-1,3-butadiene derivative (W), as described by Bonner, Polymer Bulletin, 1992, p. 517:

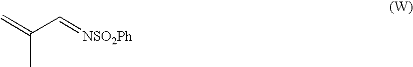

(viii) acrylate and acrylamide derivatives such as:

N-propyl-N-(3-triisopropoxysilylpropyl)acrylamide (X) and N-propyl-N-(3-triethoxysilylpropyl)acrylamide (Y), as described by Kobayashi, Journal of Polymer Science, Part A: Polymer Chemistry, 2005, p. 2754:

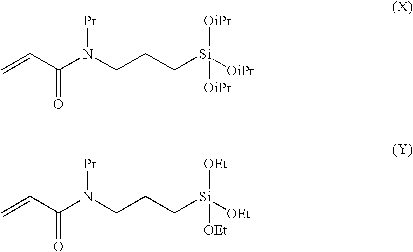

2-hydroxyethyl acrylate (Z) and 2-hydroxyethyl methacrylate (AA), as described by Rozenberg, International Journal of Plastics Technology, 2003, p.17:

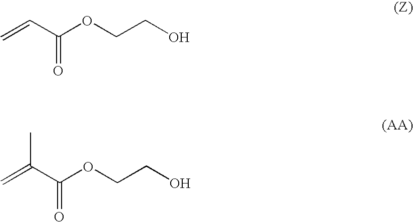

n-butyl acrylate (AB), as described by Schmitt, Macromolecules, 2001, p. 2115, and tert-butyl acrylate (AC), as described by Ishizone, Macromolecules, 1999, p. 955:

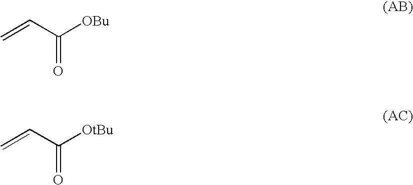

The at least one electrophilic, or electron-withdrawing, monomer useful in the cosmetic composition disclosed herein may be cyclic or linear. When it is cyclic, the electron-withdrawing group, in one embodiment, may be exocyclic, i.e., it does not form an integral part of the cyclic structure of the monomer.

In at least one embodiment, the at least one electrophiliic monomer comprises at least two electron-withdrawing groups.

Examples of the at least one electrophilic monomer comprising at least two electron-withdrawing groups include the compounds of formula (I): 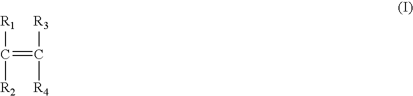
wherein:

R₁ and R₂, which may be identical or different, are each chosen from minimally or non-electron-withdrawing groups (with little or no inductive withdrawal effect), such as:
hydrogen,
saturated or unsaturated linear, branched or cyclic hydrocarbon groups, comprising, for example, from 1 to 20, further, for example, from 1 to 10 carbon atoms, and optionally comprising at least one atom chosen from nitrogen, oxygen and sulphur atoms, wherein the hydrocarbon groups may be optionally substituted by at least one group, which may be identical or different, chosen from —OR, —COOR, —COR, —SH, —SR, —OH, and halogen atoms,
modified and unmodified polyorganosiloxane residues, and
polyoxyalkylene groups and

R₃ and R₄, which may be identical or different, are each chosen from electron-withdrawing (or inductively withdrawing) groups chosen, for example, from —N(R)₃⁺, —S(R)₂⁺, —SH₂⁺, —NH₃⁺, —NO₂, —SO₂R, —C≡N, —COOH, —COOR, —COSR, —CONH₂, —CONHR, —F, —Cl, —Br, —I, —OR, —COR, —SH, —SR, —OH, linear or branched alkenyl groups, linear or branched alkynyl groups, C₁-C₄ mono- or polyfluoroalkyl groups, aryl groups such as phenyl, and aryloxy groups such as phenyloxy,

wherein R is chosen from saturated or unsaturated linear, branched or cyclic hydro-carbon groups comprising, for example, from 1 to 20 and further, for example, from 1 to 10 carbon atoms, and optionally comprising at least one atom chosen from nitrogen, oxygen and sulphur atoms, and wherein R may be optionally substituted with at least one group chosen from —OR′, —COOR′, —COR′, —SH, —SR′, —OH, halogen atoms, and polymer residues obtainable by free-radical polymerization, by polycondensation, or by ring opening, wherein R¹ is chosen from C₁-C₁₀alkyl groups.

As used herein, “electron-withdrawing or inductively withdrawing group” means any group which is more electronegative than carbon. Reference may be made to the work by P. R. Wells in Prog. Phys. Org. Chem., Vol. 6,111 (1968).

As used herein, a “minimally or non-electron-withdrawing group” is any group whose electronegativity is less than or equal to that of carbon.

In one embodiment, the alkenyl or alkynyl groups comprise, for example, from 2 to 20 carbon atoms and further, for example, from 2 to 10 carbon atoms.

In one embodiment, the saturated or unsaturated linear, branched or cyclic hydrocarbon groups comprise, for example, from 1 to 20 carbon atoms and further, for example, from 1 to 10 carbon atoms. Examples include linear or branched alkyl, alkenyl and alkynyl groups, such as methyl, ethyl, n-butyl, tert-butyl, isobutyl, pentyl, hexyl, octyl, butenyl and butynyl groups and cycloalkyl and aromatic groups.

In one embodiment, the substituted hydrocarbon groups may be chosen from hydroxyalkyl and polyhaloalkyl groups.

Examples of unmodified polyorganosiloxanes include polyalkylsiloxanes such as polydimethylsiloxanes, polyarylsiloxanes such as polyphenylsiloxanes, and polyarylalkylsiloxanes such as polymethylphenylsiloxanes.

Examples of modified polyorganosiloxanes include polydimethylsiloxanes comprising at least one group chosen from polyoxyalkylene, siloxy, silanol, amine, imine, and fluoroalkyl groups.

Examples of polyoxyalkylene groups include polyoxyethylene groups and polyoxypropylene groups comprising, for example, from 1 to 200 oxyalkylene units.

Among mono- or polyfluoroalkyl groups mention may be made, for example, of groups such as —(CH₂)n—(CF₂)m—CF₃ and —(CH₂)n—(CF₂)m—CHF₂, wherein n ranges from 1 to 20 and m ranges from 1 to 20.

The substituents R₁, R₂, R₃, and R₄ may optionally be substituted by at least one group that has cosmetic activity. The cosmetic activities may, for example, be obtained from groups having coloring, antioxidant, UV filter, or conditioning functions.

Examples of groups having a coloring function include, for example, azo, quinone, methine, cyanomethine and triarylmethane groups.

Examples of groups having an antioxidant function include, for example, butylated hydroxyanisole (BHA) groups, butylated hydroxytoluene (BHT) groups, and vitamin E groups.

Examples of groups having a UV filter function include, for example, benzophenone, cinnamate, benzoate, benzylidenecamphor, and dibenzoylmethane groups.

Examples of groups having a conditioning function include, for example, cationic groups and fatty ester groups.

In one embodiment, the at least one electrophilic monomer is chosen from cyanoacrylates of formula (II) and derivatives thereof: 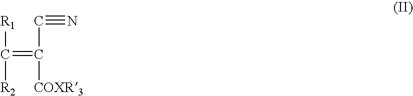
wherein:

X is chosen from NH, S, and O;

- R₁ and R₂, which may be identical or different, are each chosen from minimally and non-electron-withdrawing groups (with little or no inductive withdrawal effect), such as:
hydrogen,
saturated or unsaturated, linear, branched or cyclic hydrocarbon groups, comprising, for example, from 1 to 20, further, for example, from 1 to 10 carbon atoms, and optionally comprising at least one atom chosen from nitrogen, oxygen and sulphur atoms, wherein the hydrocarbon groups are optionally substituted by at least one group, which may be identical or different, chosen from —OR, —COOR, —COR, —SH, —SR, —OH, and halogen atoms,
modified and non-modified polyorganosiloxane residues, and polyoxyalkylene groups; and

R′₃ is chosen from a hydrogen atom and groups R, wherein R is chosen from saturated or unsaturated, linear, branched or cyclic hydrocarbon groups comprising, for example, from 1 to 20, further, for example, from 1 to 10 carbon atoms, and optionally comprising at least one atom chosen from nitrogen, oxygen and sulphur atoms, and wherein R may be optionally substituted by at least one group, which may be identical or different, chosen from —OR′, —COOR′, —COR′, —SH, —SR′, —OH, halogen atoms, and polymer residues obtainable by free-radical polymerization, by polycondensation, or by ring opening, wherein R′ is chosen from C₁-C₁₀ alkyl groups.

In one embodiment, X is an O atom.

As compounds of formula (II), mention may be made of the following monomers:

a) C₁-₂₀ polyfluoroalkyl 2-cyanoacrylates, such as:

the 2,2,3,3-tetrafluoropropyl ester of 2-cyano-2-propenoic acid, of formula:

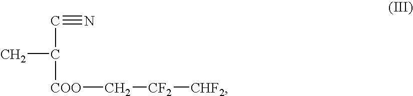

and the 2,2,2-trifluoroethyl ester of 2-cyano-2-propenoic acid, of formula:

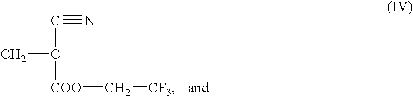

b) C₁-C₁₀ alkyl or (C₁-C₄)alkoxy(C₁-C₁₀)alkyl cyanoacrylates.

Mention may be made, for example, of ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate, and isoamyl cyanoacrylate.

In one embodiment, monomers b) may be used.

In another embodiment, the at least one electrophilic monomer may be chosen from those of formula (V):

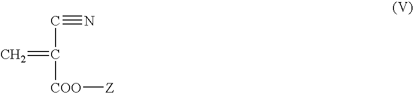

wherein: Z is chosen from ═—(CH₂)₇—CH₃, —CH(CH₃)—(CH₂)₅—CH₃, —CH₂—CH(C₂H₅)—(CH₂)₃—CH₃, —(CH₂)₅—CH(CH₃)—CH₃, and —(CH₂)₄—CH(C₂H₅)—CH₃.

The at least one electrophilic monomer disclosed herein may be attached covalently to at least one support, chosen, for example, from polymers, oligomers, and dendrimers. The polymer or oligomer may be of linear, branched, comb or block structure. The distribution of the at least one electrophilic monomer over the polymeric, oligomeric or dendritic structure may be random, terminal or blockwise.

According to one embodiment, the at least one electrophilic monomer is chosen, for example, from monomers capable of undergoing polymerization on keratin fibers under cosmetically acceptable conditions. For example, the polymerization of the at least one electrophilic monomer can take place, for example, at a temperature less than or equal to 80° C., for example, ranging from 10 to 80° C., further, for example, from 20 to 80° C., which does not prevent application being ended with an operation of drying under a hood, blow-drying or passage of a flat iron or curling tongs.

As used herein, “at least one cosmetically acceptable medium” means a medium which is compatible with the hair.

In some embodiments, the at least one cosmetically acceptable medium is anhydrous. As used herein, “anhydrous medium” means a medium comprising no more than 1% by weight of water, relative to the total weight of the composition.

The at least one cosmetically acceptable medium may, for example, be chosen from organic oils; silicones such as volatile silicones, amino and non-amino silicone gums and oils and mixtures thereof; mineral oils; vegetable oils such as olive oil, castor oil, colza oil, copra oil, wheatgerm oil, sweet almond oil, avocado oil, macadamia oil, apricot oil, safflower oil, candlenut oil, false flax oil, tamanu oil and lemon oil; waxes; and organic compounds such as C₅-C₁₀ alkanes, acetone, methyl ethyl ketone, esters of C₁-C₂₀ acids and C₁-C₈ alcohols such as methyl acetate, butyl acetate, ethyl acetate and isopropyl myristate, dimethoxyethane, diethoxyethane, C₁₀-C₃₀ fatty alcohols such as lauryl alcohol, cetyl alcohol, stearyl alcohol and behenyl alcohol, C₁₀-C₃₀ fatty acids such as lauric acid and stearic acid, C₁₀-C₃₀ fatty amides such as lauric diethanolamide, and C₁₀-C₃₀ fatty alcohol esters such as C₁₀-C₃₀ fatty alcohol benzoates, and mixtures thereof.

In one embodiment, the organic compounds are chosen from compounds which are liquid at a temperature of 25° C. and under a pressure of 10⁵ Pa (760 mm Hg).

The at least one electrophilic monomer disclosed herein may be present in the cosmetic composition disclosed herein in an amount ranging from 0.001% to 80% by weight, further, for example, from 0.1% to 40%, and, even further, for example, from 1% to 20% by weight, relative to the total weight of the cosmetic composition.

It is also possible to introduce at least one polymerization inhibitor into the cosmetic composition, for example, at least one polymerization inhibitor chosen from free-radical and anionic polymerization inhibitors, in order to increase the stability of the composition over time. For example, the at least one polymerization inhibitor may be chosen from sulphur dioxide, nitric oxide, lactone, boron trifluoride, hydroquinone and derivatives thereof, such as hydroquinone monoethyl ether, tert-butylhydroquinone (TBHQ), benzoquinone and derivatives thereof such as duroquinone, catechol and derivatives thereof such as tert-butylcatechol and methoxycatechol, anisole and derivatives thereof such as methoxyanisole, hydroxyanisole and butylated hydroxyanisole, pyrogallol, 2,4-dinitrophenol, 2,4,6-trihydroxybenzene, p-methoxyphenol, hydroxybutyltoluene, alkyl sulphates, alkyl sulphites, alkyl sulphones, alkyl sulphoxides, alkyl sulphides, mercaptans, and 3-sulpholene, and mixtures thereof. In one embodiment, the alkyl groups are chosen from groups comprising from 1 to 6 carbon atoms.

It is also possible to use organic or inorganic acids, the organic acids comprising at least one group chosen from carboxylic and sulphonic groups, which have a pKa ranging from 0 to 6, such as phosphoric acid, hydrochloric acid, nitric acid, benzene- and toluene-sulphonic acid, sulphuric acid, carbonic acid, hydrofluoric acid, acetic acid, formic acid, propionic acid, benzoic acid, mono-, di- and trichloroacetic acids, salicylic acid, and trifluoroacetic acid.

The at least one polymerization inhibitor may be present in an amount ranging from 10 ppm to 20% by weight, and, further, for example, from 10 ppm to 5% by weight, and, even further, for example, from 10 ppm to 1% by weight, relative to the total weight of the composition.

The compositions disclosed herein may further comprise at least one additive which is commonly used in cosmetic compositions, for example, reducing agents; fats; plasticizers; softeners; antifoams; moisturizers; pigments; clays; mineral fillers; UV filters; mineral colloids; peptizers; solubilizers; perfumes; preservatives; cationic, anionic, nonionic and amphoteric surfactants; fixative and non-fixative polymers; polyols; proteins; vitamins; direct dyes and oxidation dyes; pearlizers; and organic and inorganic thickeners such as benzylidenesorbitol and N-acylamino acids.

In some embodiments, the at least one additive may be optionally encapsulated. In one embodiment, the capsule may be polycyanoacrylate.

In some embodiments, the two-compartment aerosol device comprises an outer aerosol can comprising an inner pocket hermetically welded to a valve. The cosmetic composition is introduced into the inner pocket and at least one compressed gas is introduced between the pocket and the can at a pressure sufficient to make the cosmetic composition exit in the form of a spray through the orifice of a nozzle. Such a device is sold under the name EP Spray by the company EP-Spray System SA.

The cosmetic composition contained in the two-compartment aerosol device disclosed herein may be used for treating the hair, for example, for hairstyling. In one embodiment, the cosmetic composition is sprayed onto the hair in the presence of at least one nucleophilic agent.

The method for cosmetically treating hair comprises spraying a cosmetic composition as defined above, contained in the two-compartment aerosol device, onto the hair, in the presence of at least one nucleophile as defined herein.

The at least one nucleophile capable of initiating the anionic polymerization are systems known per se which are capable of generating a carbanion on contact with an electrophilic monomer. As used herein, “carbanion” means the chemical species defined in “Advanced Organic Chemistry”, Third Edition, Jerry March, page 141.

The at least one nucleophile may, for example, be chosen from molecular compounds, oligomers, dendrimers, and polymers comprising at least one nucleophilic functional group. Examples of the at least one nucleophilic functional group which may be mentioned include the following groups: R₂N⁻, NH₂⁻, Ph₃C⁻, R₃C⁻, PhNH⁻, pyridine, ArS⁻, R—C≡C⁻, RS⁻, SH⁻, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻ and H₂O, wherein Ph is a phenyl group, Ar is an aryl group and R is chosen from C₁-C₁₀ alkyl groups.

In one embodiment, the at least one nucleophile is chosen from hydroxyl ions, such as those present in water. The water may be provided by prior wetting of the hair.

In one embodiment, the cosmetic composition comprising the at least one electrophilic monomer does not comprise a nucleophile.

In yet another embodiment, a second composition comprises the at least one nucleophile. The second composition is applied to the hair before or after the first composition comprising the at least one electrophilic monomer.

It is also possible, in order to modify the reaction kinetics, to wet the hair, beforehand by means of an aqueous solution whose pH has been adjusted using at least one agent chosen from basifying and acidifying agents. The at least one agent may be organic or inorganic.

It is also possible to modify the anionic polymerization kinetics by pre-impregnating the hair, using at least one nucleophile other than water. The at least one nucleophile may be used in its pure state, in solution, in the form of an emulsion, or in the form of a capsule.

In order to modify the anionic polymerization kinetics it is also possible to enhance the nucleophilicity of the hair, by chemically converting the keratin material of the hair fiber.

By way of example of chemical conversion, mention may be made of the reduction of the disulphide bridges, of which the keratin is partly composed, to thiols, prior to application of the composition disclosed herein. Examples of reductants, i.e. compounds that can reduce the disulphide bridges to thiols, include the following:

anhydrous sodium thiosulphate,

powdered sodium metabisulphite,

thiourea,

ammonium sulphite,

thioglycolic acid,

thiolactic acid,

ammonium thiolactate,

glycerol monothioglycolate,

ammonium thioglycolate,

thioglycerol,

2,5-dihydroxybenzoic acid,

diammonium dithioglycolate,

strontium thioglycolate,

calcium thioglycolate,

zinc formaldehyde-sulphoxylate,

isooctyl thioglycolate,

dl-cysteine, and

monoethanolamine thioglycolate.

In order to modify the anionic polymerization kinetics, and more specifically to reduce the polymerization rate of the at least one electrophilic monomer, it is possible to enhance the viscosity of the composition. To accomplish this it is possible to add to the disclosed cosmetic composition at least one polymer which may exhibit no reactivity with the at least one electrophilic monomer. For example, the at least one polymer may be chosen from poly(methyl methacrylate) (PMMA) and cyanoacrylate-based copolymers as described in U.S. Pat. No. 6,224,622.

In order to improve, among other things, the adhesion of the poly(cyanoacrylate) formed in situ, it is possible to pretreat the hair fibers with many types of polymers, or to carry out a hair treatment before applying the composition disclosed herein, such as a direct dyeing, oxidation dyeing, permanent waving, or straightening treatment.

The cosmetic composition contained in the aerosol device may optionally be rinsed after it is applied.

The invention is illustrated in greater detail by the examples described below. Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters in the disclosure above are approximations, the numerical values set forth in the specific examples are reported as precisely as possible. Any numerical value, however, inherently contains certain errors necessarily resulting from the standard deviation found in its respective testing measurements.

### EXAMPLE 1

The following composition was prepared:

| | |
|---|---|
| n-Octyl 2-cyanoacrylate⁽¹⁾ | 10 g |
| Cyclopentadimethylsiloxane⁽²⁾ | 90 g |

⁽¹⁾Rite Lok CON895 sold by the company Chemence
⁽²⁾Dow Corning 245 Fluid

The composition prepared above was introduced into an aerosol distribution device sold under the name EP Spray by the company EP Spray System S.A. described above. A valve of reference 6001 format D6 is attached to a standard aerosol can, and the diffuser is a turbulent-nozzle diffuser.

The pocket was filled with the composition as indicated above. Compressed air was introduced between the pocket and the can.

The composition was sprayed onto wet hair. The spraying took place in the form of a soft spray.

After drying, hair with good hold was obtained.

### EXAMPLE 2

The following composition was prepared:

| | |
|---|---|
| Methylheptyl cyanoacrylate⁽¹⁾ | 10 g |
| Cyclopentadimethylsiloxane⁽²⁾ | 90 g |

⁽¹⁾Sold by the company Chemence
⁽²⁾Dow Corning 245 Fluid

The composition prepared above was introduced into an aerosol distribution device sold under the name EP Spray by the company EP Spray System S.A. described above. A valve of reference 6001 format D6 was attached to a standard aerosol can, and the diffuser was a turbulent-nozzle diffuser.

The pocket was filled with the composition as indicated above. Compressed air was introduced between the pocket and the can.

The composition was sprayed onto wet hair. The spraying took place in the form of a soft spray.

After drying, hair with good hold was obtained.

### EXAMPLE 3

The following composition was prepared:

| | |
|---|---|
| Methylheptyl cyanoacrylate⁽¹⁾ | 10 g |
| Cyclopentadimethylsiloxane⁽²⁾ | 89.75 g |
| Acetic acid | 0.25 g |

⁽¹⁾Sold by the company Chemence
⁽²⁾Dow Corning 245 Fluid

The composition prepared above was introduced into an aerosol distribution device sold under the name EP Spray by the company EP Spray System S.A. described above. A valve of reference 6001 format D6 was attached to a standard aerosol can, and the diffuser was a turbulent-nozzle diffuser.

The pocket was filled with the composition as indicated above. Compressed air was introduced between the pocket and the can.

The composition was sprayed onto wet hair. The spraying took place in the form of a soft spray.

After drying, hair with good hold was obtained.

### EXAMPLE 4

The following composition was prepared:

| | |
|---|---|
| Ethoxyethyl cyanoacrylate⁽¹⁾ | 10 g |
| Cyclopentadimethylsiloxane⁽²⁾ | 89 g |
| Acetic acid | 1 g |

⁽¹⁾EO 460 sold by the company Tong Shen
⁽²⁾Dow Corning 245 Fluid

The composition prepared above was introduced into an aerosol distribution device sold under the name EP Spray by the company EP Spray System S.A. described above. A valve of reference 6001 format D6 was attached to a standard aerosol can, and the diffuser was a turbulent-nozzle diffuser.

The pocket was filled with the composition as indicated above. Compressed air was introduced between the pocket and the can.

The composition was sprayed onto wet hair. The spraying took place in the form of a soft spray.

After drying, hair with good hold was obtained.

### EXAMPLE 5

The following composition was prepared:

| | |
|---|---|
| Butyl cyanoacrylate⁽¹⁾ | 10 g |
| Cyclopentadimethylsiloxane⁽²⁾ | 89 g |
| Acetic acid | 1 g |

⁽¹⁾B 60 sold by the company Tong Shen
⁽²⁾Dow Corning 245 Fluid

The composition prepared above was introduced into an aerosol distribution device sold under the name EP Spray by the company EP Spray System S.A. described above. A valve of reference 6001 format D6 was attached to a standard aerosol can, and the diffuser was a turbulent-nozzle diffuser.

The pocket was filled with the composition as indicated above. Compressed air was introduced between the pocket and the can.

The composition was sprayed onto wet hair. The spraying took place in the form of a soft spray.

After drying, hair with good hold was obtained.

### EXAMPLE 6

The following composition was prepared:

| | |
|---|---|
| Ethylhexyl cyanoacrylate⁽¹⁾ | 10 g |
| Cyclopentadimethylsiloxane⁽²⁾ | 90 g |

⁽¹⁾O-60 sold by the company Tong Shen
⁽²⁾Dow Corning 245 Fluid

The composition prepared above was introduced into an aerosol distribution device sold under the name EP Spray by the company EP Spray System S.A. described above. A valve of reference 6001 format D6 was attached to a standard aerosol can, and the diffuser was a turbulent-nozzle diffuser.

The pocket was filled with the composition as indicated above. Compressed air was introduced between the pocket and the can.

The composition was sprayed onto wet hair. The spraying took place in the form of a soft spray.

After drying, hair with good hold was obtained.

### EXAMPLE 7

The following composition was prepared:

| | |
|---|---|
| Methylheptyl cyanoacrylate⁽¹⁾ | 9 g |
| Ethylhexyl cyanoacrylate⁽³⁾ | 1 g |
| Cyclopentadimethylsiloxane⁽²⁾ | 90 g |

⁽¹⁾Sold by the company Chemence
⁽²⁾Dow Corning 245 Fluid
⁽³⁾O-60 sold by the company Tong Shen

The composition prepared above was introduced into an aerosol distribution device sold under the name EP Spray by the company EP Spray System S.A. described above. A valve of reference 6001 format D6 is attached to a standard aerosol can, and the diffuser was a turbulent-nozzle diffuser.

The pocket was filled with the composition as indicated above. Compressed air was introduced between the pocket and the can.

The composition was sprayed onto wet hair. The spraying took place in the form of a soft spray.

After drying, hair with good hold was obtained.

### EXAMPLE 8

The following composition was prepared:

| | |
|---|---|
| Methylheptyl cyanoacrylate⁽¹⁾ | 7 g |
| Butyl cyanoacrylate⁽³⁾ | 3 g |
| Cyclopentadimethylsiloxane⁽²⁾ | 90 g |

⁽¹⁾Sold by the company Chemence
⁽²⁾Dow Corning 245 Fluid
⁽³⁾B-60 sold by the company Tong Shen

The composition prepared above was introduced into an aerosol distribution device sold under the name EP Spray by the company EP Spray System S.A. described above. A valve of reference 6001 format D6 was attached to a standard aerosol can, and the diffuser was a turbulent-nozzle diffuser.

The pocket was filled with the composition as indicated above. Compressed air was introduced between the pocket and the can.

The composition was sprayed onto wet hair. The spraying took place in the form of a soft spray.

After drying, hair with good hold was obtained.

What is claimed is:

1. A two-compartment aerosol device comprising:
(i) a first compartment comprising a cosmetic composition comprising, in at least one cosmetically acceptable medium, at least one electrophilic monomer, and
(ii) in a second compartment, at least one compressed gas,
wherein the first and second compartments provide, during the use of the device, a permanent mechanical separation therein between the cosmetic composition and the at least one compressed gas.

2. The aerosol device according to claim 2, wherein the at least one compressed gas is chosen from air, nitrogen, carbon dioxide, and mixtures thereof.

3. The aerosol device according to claim 3, wherein the at least one compressed gas is air.

4. The aerosol device according to claim 1, wherein the at least one compressed gas is at a pressure ranging from 0.1 and 1.2 MPa.

5. The aerosol device according to claim 4, wherein the at least one compressed gas is at a pressure ranging from 0.9 to 1.1 MPa.

6. The aerosol device according to claim 1, wherein the at least one electrophilic monomer is chosen from compounds of formula (I): 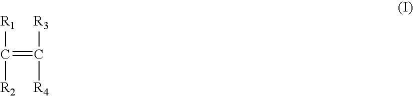
wherein:
R₁ and R₂, which may be identical or different, are each chosen from minimally and non-electron-withdrawing groups chosen from:
a hydrogen atom,
saturated or unsaturated, linear, branched or cyclic hydrocarbon groups comprising from 1 to 20 carbon atoms and comprising optionally at least one atom chosen from nitrogen, oxygen and sulphur atoms, wherein the hydrocarbon groups are optionally substituted by at least one group, which may be identical or different, chosen from —OR, —COOR, —COR, —SH, —SR, —OH and halogen atoms,
modified and unmodified polyorganosiloxane residues, and
polyoxyalkylene groups;
R₃ and R₄, which may be identical or different, are each chosen from electron-withdrawing groups chosen from —N(R)₃⁺, —S(R)₂⁺, —SH₂⁺, —NH₃⁺, —NO₂, —SO₂R, —C≡N, —COOH, —COOR, —COSR, —CONH₂—CONHR, —F, —Cl, —Br, —I, —OR, —COR, —SH, —SR, —OH, linear or branched alkenyl groups, linear or branched alkynyl groups, C₁-C₄ monofluoroalkyl groups, C₁-C₄ polyfluoroalkyl groups, and aryl and aryloxy groups, wherein R is chosen from saturated or unsaturated, linear, branched or cyclic hydrocarbon groups comprising from 1 to 20 carbon atoms and comprising optionally at least one atom chosen from nitrogen, oxygen and sulphur atoms and wherein R is optionally substituted with at least one group, which may be identical or different, chosen from —OR¹, —COOR¹, —COR¹, —SH, —SR¹, —OH, halogen atoms, and polymer residues obtained by free-radical polymerization, by polycondensation, or by ring opening, wherein R¹ is chosen from C₁-C₁₀ alkyl groups.

7. The aerosol device according to claim 6, wherein the at least one electrophilic monomer is chosen from cyanoacrylates of formula (II) and derivatives thereof: 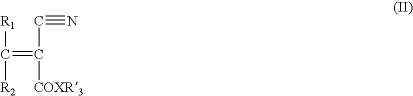
wherein:
X is chosen from NH, S, and O;
R₁ and R₂, which may be identical or different, are each chosen from minimally and non-electron-withdrawing groups chosen from:
a hydrogen atom,
saturated or unsaturated, linear, branched or cyclic hydrocarbon groups comprising from 1 to 20 carbon atoms and comprising optionally at least one atom chosen from nitrogen, oxygen and sulphur atoms, wherein the hydrocarbon groups are optionally substituted by at least one group, which may be identical or different, chosen from —OR, —COOR, —COR, —SH, —SR, —OH and halogen atoms,
modified and unmodified polyorganosiloxane residues, and
polyoxyalkylene groups and
R′₃ is chosen from a hydrogen atom and from groups R, wherein R is chosen from saturated or unsaturated, linear, branched or cyclic hydrocarbon groups comprising from 1 to 20 carbon atoms and comprising optionally at least one atom chosen from nitrogen, oxygen and sulphur atoms, and wherein R is optionally substituted by at least one group, which may be identical or different, chosen from —OR′, —COOR′, —COR′, —SH, —SR′, —OH, halogen atoms, and polymer residues obtained by free-radical polymerization, by polycondensation, or by ring opening, wherein R′ is chosen from C₁-C₁₀ alkyl groups.

8. The aerosol device according to claim 7, wherein the at least one electrophilic monomer is chosen from C₁-₂₀ polyfluoroalkyl 2-cyanoacrylates, (C₁-C₁₀ alkyl) cyanoacrylates, and (C₁-C₄ alkoxy)(C₁-C₁₀ alkyl) cyanoacrylates.

9. The aerosol device according to claim 8, wherein the at least one electrophilic monomer is chosen from ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate, and isoamyl cyanoacrylate.

10. The aerosol device according to claim 9, wherein the at least one electrophilic monomer is chosen from compounds of formula (V): 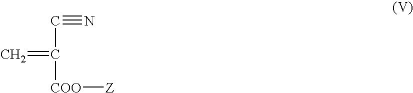
wherein Z is chosen from:
—(CH₂)₇—CH₃,
—CH(CH₃)—(CH₂)₅—CH₃,
—CH₂—CH(C₂H₅)—(CH₂)₃—CH₃,
—(CH₂)₅—CH(CH₃)—CH₃, and
—(CH₂)₄—CH(C₂H₅)—CH₃.

11. The aerosol device according to claim 1, wherein the at least one electrophilic monomer is attached covalently to at least one support.

12. The aerosol device according to claim 11, wherein the at least one support is chosen from polymers, oligomers, and dendrimers.

13. The aerosol device according to claim 1, wherein the at least one electrophilic monomer is present in an amount ranging from 0.001 to 80% by weight, relative to the total weight of the cosmetic composition.

14. The aerosol device according to claim 13, wherein the at least one electophilic monomer is present in an amount ranging from 0.1 to 40% by weight, relative to the total weight of the cosmetic composition.

15. The aerosol device according to claim 1, wherein the at least one cosmetically acceptable medium is anhydrous.

16. The aerosol device according to claim 15, wherein the at least one cosmetically acceptable medium is chosen from organic oils, silicones, mineral oils, vegetable oils, waxes, C₅-C₁₀ alkanes, acetone, methyl ethyl ketone, esters of C₁-C₂₀ acids and C₁-C₈ alcohols, dimethoxyethane, diethoxyethane, C₁₀-C₃₀ fatty alcohols, C₁₀-C₃₀ fatty acids, C₁₀-C₃₀ fatty amides, C₁₀-C₃₀ fatty alcohol esters, and mixtures-thereof.

17. The aerosol device according to claim 1, wherein the cosmetic composition comprises at least one polymerization inhibitor.

18. The aerosol device according to claim 17, wherein the at least one polymerization inhibitor is chosen from free-radical and anionic polymerization inhibitors.

19. The aerosol device according to claim 18, wherein the at least one polymerization inhibitor is chosen from sulphur dioxide, nitric oxide, lactone, boron trifluoride, hydroquinone and derivatives thereof, tert-butylhydroquinone (TBHQ), benzoquinone and derivatives thereof, catechol and derivatives thereof, anisole and derivatives thereof, pyrogallol, 2,4-dinitrophenol, 2,4,6-trihydroxybenzene, p-methoxyphenol, hydroxybutyltoluene, alkyl sulphates, alkyl sulphites, alkyl sulphones, alkyl sulphoxides, alkyl sulphides, mercaptans, 3-sulpholene, and mixtures thereof.

20. The aerosol device according to claim 17, wherein the at least one polymerization inhibitor is present in an amount ranging from 10 ppm to 20% by weight, relative to the total weight of the cosmetic composition.

21. The aerosol device according to claim 1, wherein the cosmetic composition further comprises at least one additive chosen from reducing agents, fats, plasticizers, softeners, antifoams, moisturizers, pigments, clays, mineral fillers, UV filters, mineral colloids, peptizers, solubilizers, perfumes, preservatives, anionic, cationic, nonionic and amphoteric surfactants, fixative and non-fixative polymers, polyols, proteins, vitamins, direct dyes and oxidation dyes, pearlizers, and organic and inorganic thickeners.

22. The aerosol device according to claim 21, wherein the at least one agent is encapsulated.

23. A method for cosmetically treating keratin materials comprising applying, to the keratin materials, a cosmetic composition contained in a two-compartment aerosol device, wherein the two-compartment aerosol device comprises:
(i) a first compartment comprising the cosmetic composition comprising, in at least one cosmetically acceptable medium, at least one electrophilic monomer, and
(ii) a second compartment comprising at least one compressed gas,
wherein the first and second compartments provide, during the use of the device, a permanent mechanical separation therein between the cosmetic composition and the at least one compressed gas.

24. The method according to claim 23, wherein the keratin materials are treated in the presence of at least one nucleophile.

25. The method according to claim 24, wherein the at least one nucleophile is chosen from molecular compounds, oligomers, dendrimers, and polymers comprising at least one nucleophilic functional group chosen from: R₂N⁻, NH₂⁻, Ph₃C⁻, R₃C⁻, PhNH⁻, pyridine, ArS⁻, R—C≡C⁻, RS⁻, SH⁻, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻ and H₂O, wherein Ph is a phenyl group, Ar is an aryl group and R is chosen from C₁-C₁₀ alkyl groups.

26. The method according to claim 24, wherein the at least one nucleophile is chosen from hydroxyl ions.

27. The method according to 26, wherein the hydroxyl ions are those present in water.

28. A method for styling hair comprising applying to the hair a cosmetic composition contained in a two-compartment aerosol device, wherein the two-compartment aerosol device comprises:
(i) a first compartment comprising the cosmetic composition comprising, in at least one cosmetically acceptable medium, at least one electrophilic monomer, and
(ii) a second compartment comprising at least one compressed gas,
wherein the first and second compartments provide, during the use of the device, a permanent mechanical separation therein between the cosmetic composition and the at least one compressed gas.

29. A method for cosmetically treating hair comprising spraying onto the hair, in the presence of at least one nucleophile, a cosmetic composition contained in a two-compartment aerosol device, wherein the two-compartment aerosol device comprises:
(i) a first compartment comprising the cosmetic composition comprising, in at least one cosmetically acceptable medium, at least one electrophilic monomer, and
(ii) in a second compartment, at least one compressed gas,
wherein the first and second compartments provide, during the use of the device, a permanent mechanical separation therein between the cosmetic composition and the at least one compressed gas.

30. The method according to claim 29, wherein the at least one nucleophile is chosen from molecular compounds, oligomers, dendrimers, and polymers possessing at least one nucleophilic functional group chosen from: R₂N⁻, NH₂⁻, Ph₃C⁻, R₃C⁻, PhNH⁻, pyridine, ArS⁻, R—C≡C⁻, RS⁻, SH⁻, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻ and H₂O, where Ph is a phenyl group, Ar is an aryl group, and R is chosen from C₁-C₁₀ alkyl groups.

31. The method according to claim 29, wherein the at least one nucleophile is chosen from hydroxyl ions.

32. The method according to 31, wherein the hydroxyl ions are those present in water.

33. The method according to claim 29, further comprising, before spraying the cosmetic composition of the two-compartment aerosol device on the hair,
adjusting the pH of an aqueous solution with at least one agent chosen from basifying agents and acidifying agents and
wetting the hair with the aqueous solution.

34. The method according to claim 29, wherein the hair is pre-impregnated with at least one nucleophile other than water.

35. The method according to claim 29, wherein the hair is pre-reduced before the cosmetic composition is sprayed onto the hair.

36. The method according to claim 29, further comprising rinsing the hair after spraying the cosmetic composition of the two-compartment device on the hair.
